(19) United States
(12) Patent Application Publication
(10) Pub. No.: US 2004/0073267 A1
(43) Pub. Date: Apr. 15, 2004

(54) Micro-generator implant

(76) Inventor: Asher Holzer, Haifa (IL)

Correspondence Address: DR. MARK FRIEDMAN LTD., C/o Bill Polkinghorn, Discovery Dispatch, 9003 Florin Way, Upper Marlboro, MD, 20772, US

(21) Appl. No.: 10/266,681

(22) Filed: Oct. 9, 2002

(51) Int. Cl. A61N 1/08

(52) U.S. Cl. 607/35

(57) ABSTRACT

A micro-generator implant device including: (a) a micro-generator, disposed within a living body, the micro-generator including: (i) a first mechanism for harnessing mechanical energy from a natural body movement, and (ii) a second mechanism for converting the mechanical energy to electrical energy, the electrical energy for providing power within the living body.

### FIELD AND BACKGROUND OF THE INVENTION

The present invention relates to an Internal Energy Source (IES) for powering an implant or an energy storage unit thereof, and in particular, to a micro-generator implant for providing power within a living body.

Many implantable medical devices, such as pacemakers and defibrillators, require an electrical energy source. In pacemakers and defibrillators, this energy source normally is provided by a battery pack that is contained within the implanted device. Although rechargeable batteries have been successfully employed in a variety of applications, some present day pacemakers and defibrillators use non-rechargeable batteries.

Surgery, with its attendant risks, discomforts, and cost is required when it becomes necessary to replace an implanted medical device. Because the batteries are hermetically sealed within the implanted device, the entire medical device must be surgically replaced if the batteries become depleted. To avoid or postpone surgery, it thus would be beneficial to provide longer lasting implantable devices. Longer life for an implant can be achieved by using a larger battery, however, this undesirably increases the size of the implant.

Despite the prominence of non-rechargeable batteries for powering implanted medical devices, some situations favor the use of rechargeable batteries. Some implanted medical devices, such as ventricular assist devices, require large amounts of electrical power to operate. Such devices often are powered by an external, non-implanted power source with direct electrical connection through the skin to the implant or indirectly via induction coils. It is often desirable, however, to detach the external power source from the implant, for example, when the patient bathes. During the time that the external power source is detached, the implanted device operates from battery power. Because of the large energy demand of some such implanted devices, it would be desirable to provide a rechargeable battery source for the implant to avoid having to surgically intervene to replace the non-rechargeable batteries once they become depleted. Upon reconnecting the external power source, the internal rechargeable battery pack could be recharged.

In applications in which rechargeable batteries are employed, a system to recharge the batteries is necessary. Such a recharging system should be non-invasive or minimally invasive. Several recharging techniques have been attempted.

One such technique uses direct electrical connections between an external power source and an implanted receptacle. For example, U.S. Pat. No. 4,941,472 to Moden, et al. describes an implanted electrical access port to provide a receptacle for receiving needle electrodes. The electrical access port in Moden is electrically interconnected to an implanted medical device. L-shaped needle electrodes of Moden are inserted through the patient's skin and body tissue and inserted into opposite ends of the access port. Similarly, U.S. Pat. No. 5,205,286 to Soukup, et al., discloses a subcutaneous data port that provides a plurality of conductive ports for receiving needle electrodes. Multiple needle sticks are required to mate the needles with all of the conductive ports, thus potentially increasing discomfort to the patient.

Another technique for recharging a battery of an implanted device involves transcutaneous energy transmission, a technique that allows non-invasive battery charging. Using transcutaneous energy transmission, such as described in U.S. Pat. No. 5,411,537, an alternating current (AC) in an external primary coil of wire creates a magnetic field, which, in turn, induces an AC electrical current in a secondary coil of wire that is housed within the implanted medical device. Charging energy is thus transmitted in the same manner as between the primary and secondary coils of a transformer. The alternating current induced in the implanted secondary coil is then rectified and regulated to provide direct current (DC) power for charging the medical device's battery. This technique advantageously recharges the battery non-invasively.

Transcutaneous energy transmission, although generally safe and reliable, is not without certain shortcomings. For example, the efficiency of transcutaneously inducing a current in the implanted coil is detrimentally affected if the internal and external coils are not properly aligned or oriented, or if the distance between the external and internal coils is too great. Because there is no direct physical connection between the external charger and the implanted device to provide feedback, ascertaining whether transmission efficiency is maximized or whether the battery has become fully charged is problematic.

Also, as mentioned previously, transcutaneous energy transmission relies upon a magnetic field to induce an AC current in the implanted coil. At the same time, the alternating magnetic flux generated by the AC current induces the formation of eddy currents in the medical device's metal housing and in the metal casings of various components internal to the implantable device. The magnitude of these eddy currents is a function of the frequency and magnitude of the magnetic flux. Eddy currents cause a temperature increase in the metal components in which the current is conducted. If too great, the temperature increase in the implanted device caused by eddy currents can damage the surrounding body tissues. A high charging current, moreover, creates large temperature rises, thereby increasing the risk of harm to surrounding tissues.

To minimize patient discomfort, it is desirable that the implanted device and all its components be as small as possible. Unfortunately, because of the inefficiency associated with electromagnetic induction, it has been necessary to employ relatively large coils in conventional transcutaneous energy transmission schemes. A relatively large size for the internal coil causes the implanted medical device to be significantly larger than the device would otherwise need to be, and thus is not consistent with the design goal of producing smaller and lighter implantable devices.

U.S. Pat. No. 5,205,286 to Schroeppel, et al., discloses an energy transmission system is provided for transmitting energy non-invasively from an external charging unit to an implanted medical device to recharge a battery in the medical device. An alternating magnetic field is generated by the external unit. One or more piezoelectric devices in the implanted medical device vibrate in response to the magnetic flux, thereby generating an AC voltage, which is rectified and regulated to provide charging current to a rechargeable battery in the medical device. An alignment indicator is provided to ascertain the optimal orientation between the external unit and the implanted medical device. The piezoelectric devices are relatively small and thin and thus reduce packaging limitation problems caused by coils of wire used in conventional transcutaneous energy transmission systems.

Alternatively, acoustic waves generated by the external charging unit can be used to vibrate the piezoelectric device instead of a changing magnetic flux. The acoustic waves are generated by an external source coupled to a piezoelectric transducer.

U.S. Pat. No. 6,432,050 to Porat, et al., teaches an implantable biosensor system for monitoring and optionally alleviating a physiological condition in a patient. The system includes (a) a sensor for sensing a parameter of a physiological condition and for generating electrical sensor signals representative of the physiological condition; and (b) a first acoustic activatable transducer, coupled with the sensor, for converting a received acoustic interrogation signal from outside the patient's body into electrical power for energizing the processor, and for converting the electrical sensor signals of the sensor into acoustic signals receivable out of the patient's body, such that information pertaining to the physiological condition parameter can be relayed outside the patient's body upon generation of an acoustic interrogation signal. U.S. Pat. No. 6,432,050 explicitly discloses the conversion of a received acoustic interrogation signal from outside the body of the patient into electrical power for energizing the processor.

In summary, implants utilizing non-rechargeable batteries have severe shortcomings, not the least of which is the need for surgically intervention to replace the depleted batteries. Some recharging systems utilize direct electrical connections that require unwieldy implants and may cause patient discomfort. All known systems and methods for non-invasively recharging implanted batteries have associated limitations, inefficiencies, and medical complications and/or risk. Finally, the improvements in battery technologies notwithstanding, rechargeable batteries lose their rechargeability over time and with increasing number of recharge cycles.

There is therefore a recognized need for, and it would be highly advantageous to have, a system and method of powering an implanted medical device that overcomes these and other problems associated with the various conventional systems. It would be of further advantage to have a system that minimizes the size of components internal to the implanted device necessary for powering the device, such that the overall size of the implanted device is reduced. It would be of yet further advantage for such a system, in powering the implanted medical device, would reduce risk and discomfort to the patient, with respect to known systems. Despite the substantial advantages that would be afforded by such a system, to date, no such system has been developed.

### SUMMARY OF THE INVENTION

The present invention is a micro-generator implant for providing power within a living body. The power is generated within the body, from the natural motion of the body, including a natural motion of an internal organ like the heart.

According to the teachings of the present invention there is provided a micro-generator implant device including: (a) a micro-generator, disposed within a living body, the micro-generator including: (i) a first mechanism for harnessing mechanical energy from a natural body movement, and (ii) a second mechanism for converting the mechanical energy to electrical energy, the electrical energy for providing power within the living body.

According to another aspect of the present invention there is provided a method of generating energy within a living body, the method including the steps of: (a) providing a micro-generator implant device including: (i) a micro-generator, disposed within a living body, the micro-generator including: (A) a first mechanism for harnessing mechanical energy from a natural body movement, and (B) a second mechanism for converting the mechanical energy to electrical energy, and (b) generating energy within the living body by means of the micro-generator.

According to still further features in the described preferred embodiments, the electrical energy converted by the second mechanism is for providing power to an implant within the living body.

According to still further features in the described preferred embodiments, the device further includes: (b) an energy storage unit, operatively connected to the second mechanism, for storing the electrical energy, the energy storage unit being operatively connected with the implant.

According to still further features in the described preferred embodiments, the device further includes: (b) an implant, operatively connected to the second mechanism, such that the second mechanism provides the electrical energy to the implant.

According to still further features in the described preferred embodiments, the energy storage unit includes a rechargeable battery.

According to still further features in the described preferred embodiments, the energy storage unit includes a capacitor.

According to still further features in the described preferred embodiments, the implant includes a pacemaker.

According to still further features in the described preferred embodiments, the implant includes a defibrillator.

According to still further features in the described preferred embodiments, the implant includes an internal communication device.

According to still further features in the described preferred embodiments, the implant includes an internal monitoring device.

According to still further features in the described preferred embodiments, the implant includes an intra-cardiac device.

According to still further features in the described preferred embodiments, the implant includes an intra blood vessel device.

According to still further features in the described preferred embodiments, the implant includes an intra-coronary device.

According to still further features in the described preferred embodiments, the implant includes a sub-cutaneous device.

According to still further features in the described preferred embodiments, the first mechanism is associated with a stent.

According to still further features in the described preferred embodiments, the stent is a coronary stent.

According to still further features in the described preferred embodiments, the stent is a graft stent.

According to still further features in the described preferred embodiments, the first mechanism includes a ratchet mechanism.

According to still further features in the described preferred embodiments, the first mechanism includes a magnet for inducing a magnetic field.

According to still further features in the described preferred embodiments, the first mechanism includes a shaft disposed within a coil, the shaft and the coil designed and configured to move within the magnetic field so as to produce a changing magnetic flux through the coil.

According to still further features in the described preferred embodiments, the second mechanism includes a dynamo for generating current.

According to still further features in the described preferred embodiments, the natural body movement is a displacement of heart tissue.

According to still further features in the described preferred embodiments, the natural body movement is a twisting motion produced during a beating of the heart.

According to still further features in the described preferred embodiments, the displacement is a substantially-linear displacement.

According to still further features in the described preferred embodiments, the natural body movement includes a flowing of blood through a blood vessel.

According to still further features in the described preferred embodiments, the first mechanism includes a rotating mechanism for rotating within the blood vessel.

According to still further features in the described preferred embodiments, the first mechanism includes a leaf for absorbing energy from the flowing of blood through the blood vessel.

According to still further features in the described preferred embodiments, the first mechanism includes a flexible membrane, operatively connected to a wall of the blood vessel, for absorbing energy from the flowing of blood through the blood vessel.

According to still further features in the described preferred embodiments, the flexible membrane is disposed within the blood vessel.

According to still further features in the described preferred embodiments, the second mechanism is disposed outside of the blood vessel.

According to still further features in the described preferred embodiments, the micro-generator is disposed within a limb of the living body.

According to still further features in the described preferred embodiments, the limb includes a prosthesis device.

According to still further features in the described preferred embodiments, the micro-generator is operatively connected with the stent, the stent disposed within a blood vessel.

According to still further features in the described preferred embodiments, the stent is a cylindrical stent.

According to still further features in the described preferred embodiments, the stent is a coil stent.

According to still further features in the described preferred embodiments, the method further includes the step of: (c) supplying energy to the micro-generator from a natural motion of the body.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing What is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1a is a block diagram of a system for converting internal body tissue motion to electrical power by means of a micro-generator so as to power a body implant;

FIG. 1b is a block diagram of the system of FIG. 1a, in which the micro-generator charges an energy storage unit for powering the implant;

FIG. 2 is a schematic exploded view showing the general construction of an electronic watch having a prior-art micro-generator, as disclosed by U.S. Pat. No. 6,183,125, the micro-generator for use in conjunction with the present invention;

FIG. 3a is a schematic illustration of a micro-generator device disposed on a coronary stent;

FIG. 3b is a schematic illustration of a micro-generator device disposed on a cylindrical blood vessel stent;

FIG. 4 is a schematic illustration of a rotating device, disposed within a blood vessel, for generating energy from the motion of a blood stream;

FIG. 5 is a schematic illustration of a mechanical leaf, disposed within a blood vessel, for generating energy from the motion of a blood stream;

FIG. 6 is a schematic illustration of the inner workings for harnessing the energy from the motion of the leaf illustrated in FIG. 5;

FIG. 7 is a schematic illustration of a flexible membrane, disposed within a blood vessel, which is periodically displaced by the motion of a blood stream, and

FIG. 8 is a schematic illustration of a micro-generator, implanted within a limb of a body, according to one embodiment of the present invention.

### DESCRIPTION OF THE PREFERRED EMBODIMENTS

The present invention is a micro-generator implant for providing power within a living body.

The principles and operation of the micro-generator implant according to the present invention may be better understood with reference to the drawings and the accompanying description.

Before explaining at least one embodiment of the invention in detail, it is to be understood that the invention is not limited in its application to the details of construction and the arrangement of the components set forth in the following description or illustrated in the drawing. The invention is capable of other embodiments or of being practiced or carried out in various ways. Also, it is to be understood that the phraseology and terminology employed herein is for the purpose of description and should not be regarded as limiting.

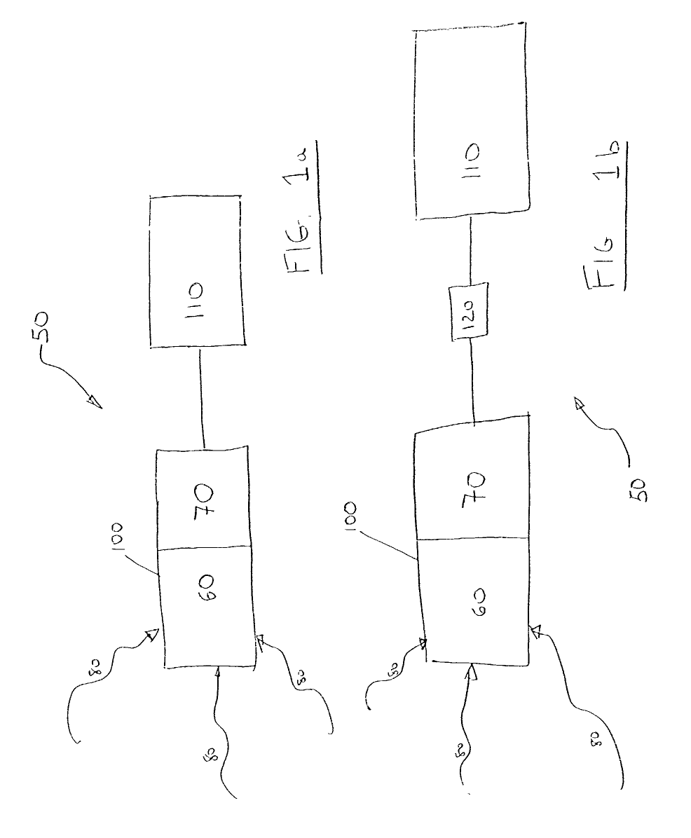

One aspect of the present invention is represented as a block diagram in FIG. 1a. In the internal power system 50, internal body tissue motion 80 is harnessed by a micro-generator 100 so as to power an implant 110. In FIG. 1b, internal body tissue motion 80 is harnessed by a micro-generator 100 so as to charge energy storage unit 120, which in turn powers implant 110.

Energy storage unit 120 is preferably selected from a wide variety of known internal energy storage units, including, but not limited to, capacitors and rechargeable batteries.

As used herein in the specification and in the claims section that follows, the term “implant” refers to any powered device implanted in the body, including, but not limited to, pacemakers, defibrillators, internal communication devices, monitoring devices such as: heart condition, heart beat, ECG, electrocardiogram, blood contents, blood pressure, temperature, blood leak from a graft stent, blood vessel ruptures, and combinations thereof. More generally, the term “implant” is meant to include intra-cardiac, intra-coronary, sub-cutaneous, and other intra-body devices. The implant may be incorporated as a part of a prosthesis device, coronary stent, blood vessel stents, etc. The term “implant” is meant to specifically include internally implanted devices that are traditionally powered by external energy sources or by batteries or other energy storage devices.

Referring back to FIG. 1a, micro-generator 100, which converts mechanical energy (internal body tissue motion) to electrical energy, is also preferably selected from a wide variety of known units, examples of which are provided hereinbelow. These units many include automatic winding systems such as those used in self-winding watches. Many known automatic winding systems include an inertia mass that oscillates concentrically with the axis of the hands of the watch when the position of the watch is being changed. The power thus obtained is transmitted to the ratchet wheel of a barrel either by a gear train or by a toothed segment, or by an intermediate lever.

In one known type of device, the inertia mass is allowed to oscillate by a fraction of a revolution (approximately 120 degrees), its movement being limited by two damper stops fixed on the plates of the watch movement; the winding only takes place in one direction of oscillation. In a second known type of device, the inertia mass is allowed to rotate freely about its axis without limitation, but the winding takes place in a single direction of oscillation. In a third known type of device, the inertia mass is allowed to turn freely about its axis without limitation of movement, and the winding is effected in both directions of oscillation. In a fourth known type of device, an eccentric crank portion on the oscillating rotor staff imparts rectilinear motion to a spring loaded double hairpin ratchet spring driving the ratchet wheel.

U.S. Pat. No. 3,104,517, which is incorporated by reference for all purposes as if fully set forth herein, discloses an automatic winding mechanism for a watch in which the motion conversion device includes a heart-shaped cam and spring loaded pawls pivotably mounted on a lever, which cooperate to actuate a ratchet wheel. The ratchet wheel winds the mainspring barrel through planetary reduction gears.

U.S. Pat. No. 4,174,607 to Wuthrich, which is incorporated by reference for all purposes as if fully set forth herein, teaches an improvement on a mainspring driving a time gear train, and having reduction gearing for periodically winding the mainspring by unidirectional rotation of a winding pinion. The improvement consists of a ratchet wheel connected to drive the pinion, a rotor bushing, a rotor staff having an eccentric crank portion disposed in the bushing, a rotor with an off-center mass connected to the rotor staff, and a single piece rocking arm with first and second spring loaded click arms engaging the ratchet wheel and a spring clip engaging the crank portion of the rotor staff and adapted to retain the rotor shaft within the bushing. The rocking arm is made from a single stamping with click arms arranged to provide spring loading of the ratchet wheel, tabs to hold the rocking arm in place and spring clip fingers inserted through a slot in the rotor bushing.

U.S. Pat. No. 6,183,125 to Hara, et al., which is incorporated by reference for all purposes as if fully set forth herein, teaches an electronic watch including a so-called automatic winding dynamo.

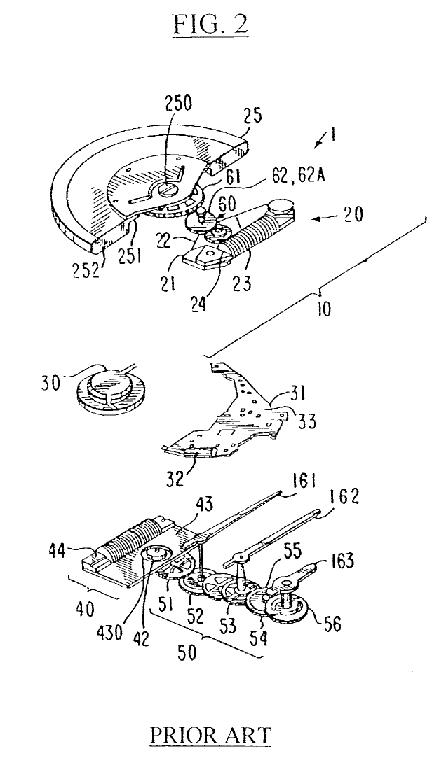

FIG. 2, which is based on FIG. 1 of the above-referenced U.S. patent to Hara, et al., is a schematic exploded view showing the general construction of an electronic watch. An electronic watch 1 is an analog quartz wrist watch. A stepping motor 40 is driven in accordance with a signal output from a crystal oscillator 32 mounted on a circuit board 31. The stepping motor 40 includes a motor rotor 42 having a permanent magnet magnetized into two poles, a motor stator 43 having a cylindrical rotor installation hole 430 in which the motor rotor 42 is disposed, and a coil block formed by winding a coil 41 over a magnetic core 44. A watch wheel train 50 including of a fifth wheel 51, a second wheel 52, a third wheel 53, a center wheel 54, a minute wheel 55 and a hour wheel 56 is operatively connected to the motor rotor 42 through respective pinions. A second hand 161 is fixed to the distal end of a shaft of the second wheel 52 of the watch wheel train. A minute hand 162 is fixed to the distal end of a cylindrical shaft of the center wheel 54. An hour hand 163 is fixed to the distal end of a cylindrical shaft of the hour wheel 56. Here, a speed reducing ratio achieved through the gearing from the motor rotor 42 to the second wheel 52 is set to 1/30. The second hand 161 is constructed such that it is intermittently rotated in steps of 6 whenever the motor rotor 42 is intermittently rotated in steps of 180 for each second.

A power supply section 10 for driving the stepping motor 40 is primarily made up of a small-sized dynamo 20 and a secondary power supply 30 (capacitor). In order to generate power upon movement of the user's wrist over which the electronic watch 1 with hands is fitted, the small-sized dynamo 20 includes an eccentric oscillating weight 25 rotatable in response to the wrist movement, a dynamo rotor 21 rotated by receiving kinetic energy from the oscillating weight 25, a dynamo stator 22 disposed in surrounding relation to the dynamo rotor 21, and a dynamo coil 23 wound over a magnetic core 24 making up a magnetic circuit in cooperation with the dynamo stator 22 and the dynamo rotor 21. The oscillating weight 25 and the dynamo rotor 21 are operatively interconnected through a dynamo wheel train 60 for transmitting rotation of the oscillating weight 25 while speeding up the rotation. The dynamo wheel train 60 is made up of a oscillating weight wheel 61 formed integrally with the oscillating weight 25, and a dynamo rotor transmitting wheel 62 having a pinion held in mesh with the oscillating weight wheel 61. The dynamo rotor 21 has a permanent magnet magnetized to have N and S poles which are rotated when the rotation of the oscillating weight 25 is transmitted to the dynamo rotor 21. Accordingly, induced electromotive force can be taken out of the dynamo coil 23 and charged into the secondary power supply 30. The oscillating weight 25 has an oscillating weight fixing screw 250 attached to its rotating central portion. The oscillating weight 25 is formed such that its inner peripheral portion around the oscillating weight fixing screw 250 (rotating central portion) provides a thinner wall portion 251 as a light oscillating weight, and its outer peripheral portion provides a thicker wall portion 252 as a heavy oscillating weight stretching radially outward from the light oscillating weight. As a result, in spite of a reduction in thickness of the oscillating weight 25, weight unbalance of the oscillating weight 25 in the angular direction remains large.

United States Patent Application No. 20020060954 to Schafroth, et al., which is incorporated by reference for all purposes as if fully set forth herein, discloses a micro-generator fitted in a watch movement. The watch movement contains a mechanical energy storage in the form of a spring. The spring is wound by a winding device or preferably by a mass that is put into oscillation by the arm movements of the watch wearer. The spring drives the various hands and displays of the watch, especially the seconds hand that is fastened on the seconds axis over a conventional gearing.

U.S. Pat. No. 6,381,198 to Born, which is incorporated by reference for all purposes as if fully set forth herein, teaches a clockwork movement fitted with a generator powering the circuit for regulating the rotation of the generator rotor. The generator is formed of at least one coil placed between magnets respectively secured to two flanges mounted at the ends of a shaft of the rotor. The coils are arranged on a substrate that includes means allowing at least one coil to be moved relative to the rotor.

Other documents incorporated by reference for all purposes as if fully set forth herein are U.S. Pat. No. 5,025,428 and U.S. Pat. No. 3,518,464.

It is evident that many alternatives, modifications and variations of the above-described micro-generator implant system will be apparent to those skilled in the art. It is also evident that various modifications to existing automatic winding systems and micro-generators could be made by one skilled in the art so as to better adapt such devices to the various applications of the present invention. The energy of the moving shaft can wind a spring and release the energy of the spring to a dynamo. Alternatively, a relative motion of a shaft and a coil surrounding the shaft, in the presence of a magnetic field, produces electrical energy. In some designs, the shaft moves within a stationary coil; in other designs, the shaft is stationary and the coil moves. The motion may be longitudinal with respect to the shaft axis or rotational. It must be emphasized that the various designs and configurations are well-known in the art, hence, there is a more detailed description herein is unnecessary.

In FIGS. 1a and 1b, micro-generator 100 includes a mechanical section 60 for harnessing the mechanical energy from a natural body movement, and a conversion section 70 in which mechanical energy from mechanical section 60 is converted to electrical energy. It must be emphasized that, while a micro-generator typically performs both the harnessing and the conversion functions in an integral unit, it is technologically feasible to implement the present invention by combining individual, stand-alone units performing a single function, so as to produce a micro-generator system that operates in vivo so to provide power to an implant.

As mentioned above, the “watch mechanism” (mechanical section 60) can either charge an electric storage unit or directly power the implanted device.

Various sources of internal mechanical energy can be utilized by the system of the present invention, including:

motion of heart muscle tissue

motion of blood passing through a blood vessel

motion of a limb, or of the entire body

I. Motion of Heart Muscle Tissue

Two types of heart motion can be utilized to generate energy: (1) a twisting motion produced during every single beat of the heart, and (2) the heart beat itself, i.e., the displacement occurring due to the contraction and expansion of the heart during every beat.

For utilization of the twisting motion of the heart, the moving shaft located within mechanical section 60 is preferably implanted in the human body in an orientation that enables the moving shaft (located inside the coil) to move back and forth due to the twisting motion resulting from the heart beat. As is known in the art, the shaft shape can be linear, or arc-shaped, so as to better utilize the energies associated with the multi-dimensional twisting motion of the heart.

For harnessing of the displacement resulting from the contraction and expansion of the heart, the device is preferably placed such that the axis of the shaft is substantially perpendicular to the heart, such that the moving shaft is moved back and forth in every single beat.

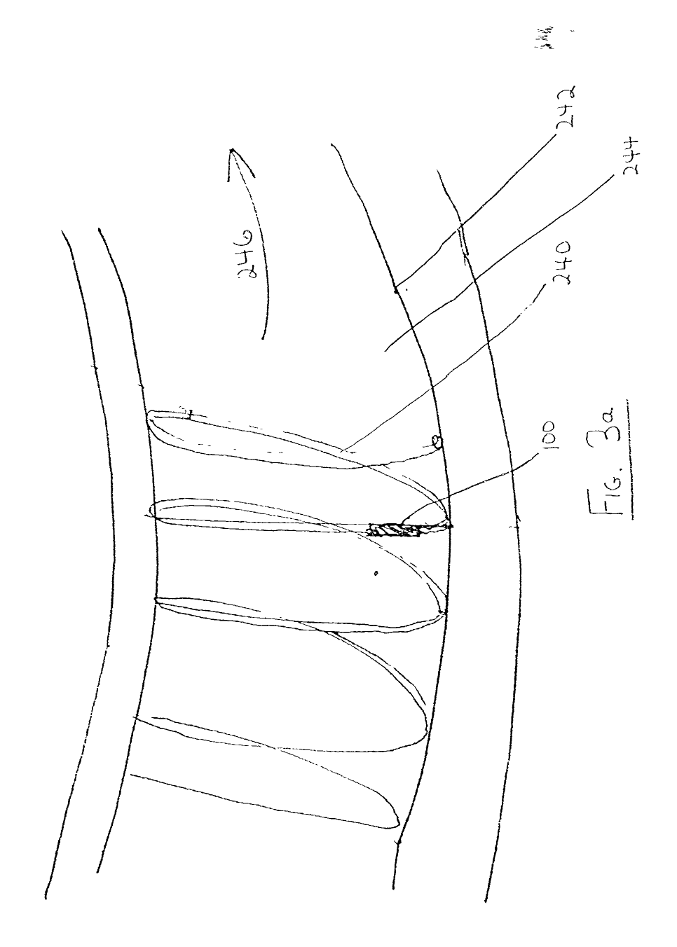

In a preferred embodiment of the present invention, the displacement resulting from the contraction and expansion of the heart is harnessed by a micro-generator device associated with, or disposed on a coronary stent. FIG. 3a is a schematic illustration of a micro-generator 100 disposed on a coronary stent 240 within an inner wall 242 of blood vessel 244. Blood stream 246 flows past micro-generator 100. The above-described displacement acts upon micro-generator 100, which is described in greater detail with reference to FIGS. 1a and 1b, thereby providing mechanical energy which is subsequently converted to electrical energy for powering an implant. Such a coronary stent may include monitoring capabilities as previously described.

Another preferred embodiment is provided schematically in FIG. 3b. The stent is a cylindrical stent 250, disposed within an inner wall 242 of blood vessel 244. A micro-generator 100a is disposed on an outer surface 252 of cylindrical stent 250, between stent 250 and inner wall 242 of blood vessel 244. Alternatively, the micro-generator is disposed within cylindrical stent 250. Micro-generator 100a, shown in FIG. 3b, has a surface 254 that may or may not be in contact with blood stream 246.

Although several exemplary devices have been described in the figures and associated text, it is evident that many alternatives, modifications and variations will be apparent to those skilled in the art.

II. Motion of Blood Passing Through a Blood Vessel

In this method, blood friction within a blood vessel is used to generate electrical power. The energy that the blood stream produces via friction with a generator device located within, or adjacent to, the blood vessel, is converted to electrical power that can either directly power any other device located there (as in FIG. 1a) or can pump its energy into a secondary energy storage device (as in FIG. 1b), that then powers another internal device.

The moving part of the generator can be either directly immersed in the blood stream, or can obtain the frictional energy through a flexible thin membrane, such that any moving parts do not directly contact the blood stream.

Direct Immersion Devices

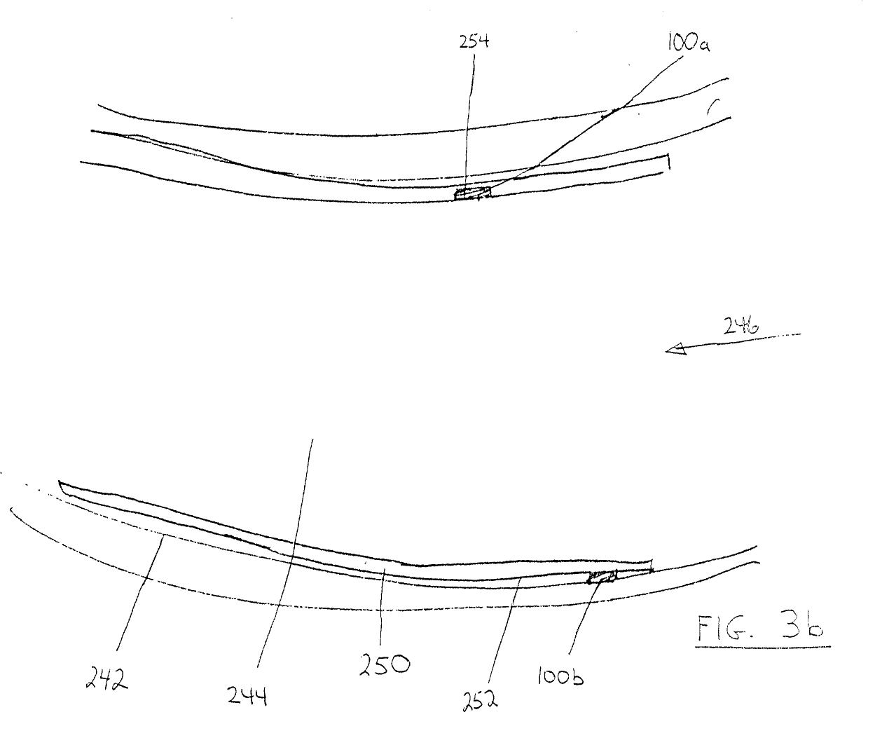

Various designs and configurations will be apparent to one skilled in the art. In FIG. 4, by way of example, a blood stream 204 within a blood vessel such as artery 206 rotates a rotor 208 having fins 210, like a water mill, thereby producing mechanical energy. Rotor 208 is held in position within artery 206 by arm 212, which is operatively connected to a wall of artery 206. The mechanical energy produced is subsequently converted into electrical energy in conversion section 70 (see FIGS. 1a, 1b).

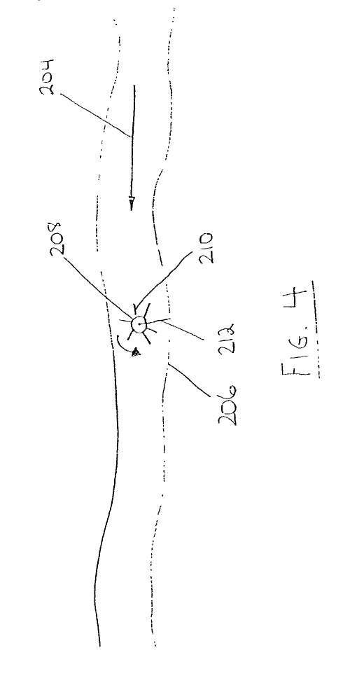

In another exemplary design, shown schematically in FIG. 5, blood stream 204 within a blood vessel such as artery 206 rotates or vibrates at least one thin leaf or plate 220, the motion of which is converted into electrical energy in conversion section 70 (see FIGS. 1a, 1b). Thin leaf or plate 220 is preferably made from a flexible membrane that has a built-in tension forcing the membrane to return to its original position during low-pressure periods. The systolic blood pressure bends plate 220 in the direction of blood stream 204, to position A, winding a spring (not shown) or working against an internal spring tension while bending plate 220, while, in the low-pressure regime of the diastolic duration, plate 220 bends back substantially to the original position (position B), urged by the internal spring tension. The motion of thin leaf or plate 220 is harnessed in gear box 231.

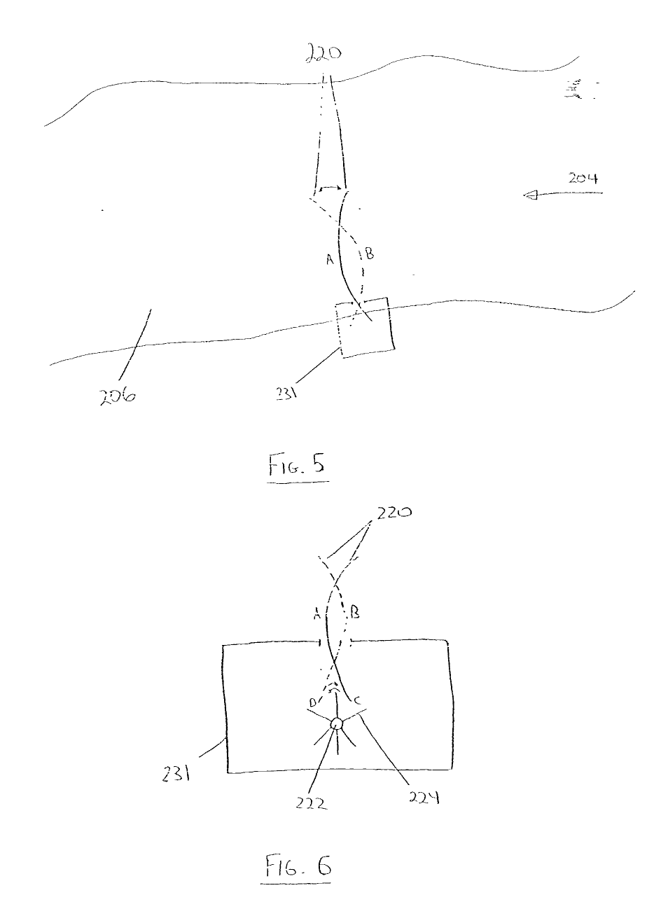

The inner workings of gear box 231 are shown schematically in FIG. 6. Plate 220 is disposed so as to make contact with or operatively connect to a wheel 222 having teeth 224 or fins disposed thereon. When the pressure within artery 206 exceeds a particular value (e.g., 90 mm Hg), plate 220 moves towards position B; in the diastolic period, plate 220 returns to position A. The movement of plate 220 from position A to B, and from B to A displaces the tip of plate 220 within gear box 231 from position C to D, and from D to C, so as to turn wheel 222. The mechanical energy produced is transferred to a shaft (not shown), which rotates correspondingly. The mechanical energy of the rotating shaft is converted to electrical energy as described hereinabove.

Non-Immersion Devices

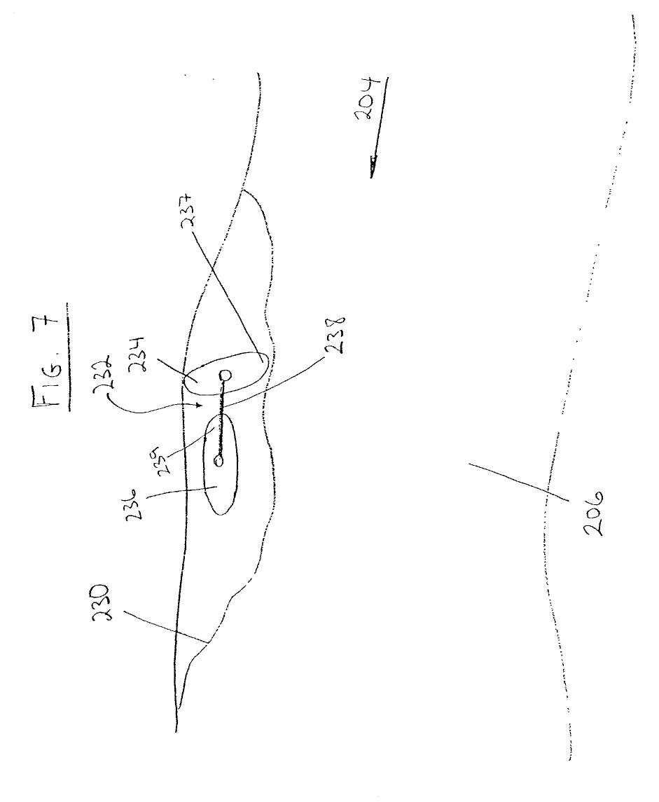

In the non-immersing configurations, blood clots and clogging of the moving parts of the mechanism by blood debris are avoided. In FIG. 7, a blood stream 204 within a blood vessel such as artery 206 produces a force on a flexible membrane 230, which is displaced by the force, particularly when the diastolic pressure within artery 206 exceeds some value. Flexible membrane 230 is further designed and configured such that on an opposing side of membrane 230, physically isolated from the interior of artery 206, the mechanical energy produced is converted into electrical energy in conversion section 70 (described hereinabove).

In one exemplary embodiment, shown in FIG. 7, the mechanism 232 disposed on the opposing side of membrane 230 is driven in a substantially peristaltic fashion by the movement of flexible membrane 230. Typically, a peristaltic pump, driven by any electrical motor or by other means, conveys the pumped fluid by periodic constriction of a membrane or tubing containing the pumped fluid. The constriction is usually performed by rotating wheels or rollers. However, instead of using electrical power to peristaltically pump a fluid, the peristaltic action can be reversed by harnessing an existing fluid flow so as to peristaltically drive a shaft (or other means), thereby generating electricity.

Peristaltic mechanism 232 includes two oval rollers 234, 236, rigidly connected by, and mounted on, connector 238. A first end of connector 238 is attached to a center of oval roller 234, and an opposite end of connector 238 is attached to a center of oval roller 236. Rollers 234, 236 are disposed at a substantially right angle to each other, such that at any given time, at least one end 237 of oval roller 234 or oval roller 236 protrudes in a perpendicular or near-perpendicular fashion with respect to blood stream 204. Consequently, and as can be seen in FIG. 7, the movement of blood stream 204 acts upon membrane 230, which in turn acts upon end 237 so as to rotate roller 234. End 237 eventually rotates enough to contact and rotate an end 239 of roller 236. Hence, a cycle occurs in which roller 234 drives roller 236, followed by roller 236 driving roller 234. The motion of oval rollers 234, 236 is utilized by conventional means to produce electricity.

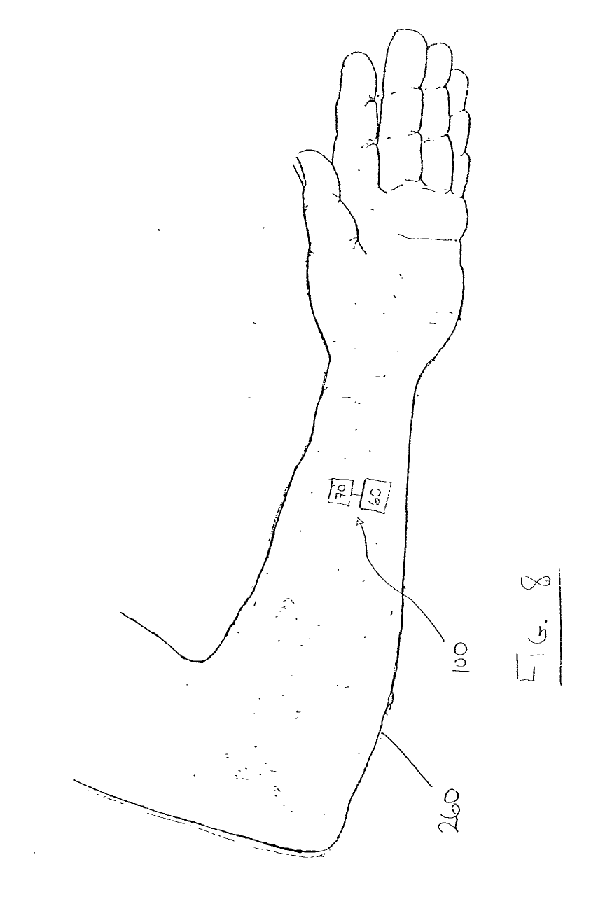

III. Motion of a Limb In FIG. 8, micro-generator 100 is implanted within a limb 260 of a user, in this case forearm 260. Mechanical section 60 harnesses the mechanical energy provided by the motions of forearm 260, and conversion section 70 converts the harnessed mechanical energy from mechanical section 60 to electrical energy. Improved efficiency in harnessing the motions of forearm 260 is attained by proper orientation of mechanical section 60 with respect to the typical motions of forearm 260. This exemplary embodiment is like “wearing” a self-winding watch within forearm 260, rather than around forearm 260.

As described hereinabove, many embodiments of mechanisms for harnessing motion are suitable for use in conjunction with the present invention. Proper or preferred orientation of mechanical section 60 depends on the specific mechanisms for harnessing the motion of the limb, and, of course, the direction or directions of the motion that are to be harnessed. For a moving shaft-type mechanism located within mechanical section 60, the moving shaft-type mechanism is preferably placed such that the axis of the shaft is substantially perpendicular to the length of forearm 260.

It should be emphasized that limb (forearm) 260 can be a prosthesis device.

Of late, various in-vivo monitoring devices, which have characteristically low power requirements, are being utilized in a wide variety of applications. The present invention is especially useful in conjunction with in-vivo monitoring devices, which, upon receiving a call or signal from a source external to the body, at a pre-scheduled time, or when some irregularity (hard attack, arrhythmia, etc.) is detected, performs a monitoring function and transmits data to an external device (e.g., a beeper, telephone, etc.).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A micro-generator implant device comprising:
(a) a micro-generator, disposed within a living body, said micro-generator including:
(i) a first mechanism for harnessing mechanical energy from a natural body movement, and
(ii) a second mechanism for converting said mechanical energy to electrical energy,
said electrical energy for providing power within said living body.

2. The micro-generator implant device of claim 1, wherein said electrical energy converted by said second mechanism is for providing power to an implant within said living body.

3. The micro-generator implant device of claim 2, further comprising:
(b) an energy storage unit, operatively connected to said second mechanism, for storing said electrical energy, said energy storage unit being operatively connected with said implant.

4. The micro-generator implant device of claim 1, further comprising:
(b) an implant, operatively connected to said second mechanism, such that said second mechanism provides said electrical energy to said implant.

5. The micro-generator implant device of claim 3, wherein said energy storage unit includes a rechargeable battery.

6. The micro-generator implant device of claim 3, wherein said energy storage unit includes a capacitor.

7. The micro-generator implant device of claim 2, wherein said implant includes a pacemaker.

8. The micro-generator implant device of claim 2, wherein said implant includes a defibrillator.

9. The micro-generator implant device of claim 2, wherein said implant includes an internal communication device.

10. The micro-generator implant device of claim 2, wherein said implant includes an internal monitoring device.

11. The micro-generator implant device of claim 2, wherein said implant includes an intra-cardiac device.

12. The micro-generator implant device of claim 2, wherein said implant includes an intra blood vessel device.

13. The micro-generator implant device of claim 2, wherein said implant includes an intra-coronary device.

14. The micro-generator implant device of claim 2, wherein said implant includes a sub-cutaneous device.

15. The micro-generator implant device of claim 2, wherein said first mechanism is associated with a stent.

16. The micro-generator implant device of claim 2, wherein said stent is a coronary stent.

17. The micro-generator implant device of claim 2, wherein said stent is a graft stent.

18. The micro-generator implant device of claim 2, wherein said first mechanism includes a ratchet mechanism.

19. The micro-generator implant device of claim 2, wherein said first mechanism includes a magnet for inducing a magnetic field.

20. The micro-generator implant device of claim 19, wherein said first mechanism includes a shaft disposed within a coil, said shaft and said coil designed and configured to move within said magnetic field so as to produce a changing magnetic flux through said coil.

21. The micro-generator implant device of claim 2, wherein said second mechanism includes a dynamo for generating current.

22. The micro-generator implant device of claim 1, wherein said natural body movement is a displacement of heart tissue.

23. The micro-generator implant device of claim 1, wherein said natural body movement is a twisting motion produced during a beating of the heart.

24. The micro-generator implant device of claim 22, wherein said displacement is a substantially-linear displacement.

25. The micro-generator implant device of claim 1, wherein said natural body movement includes a flowing of blood through a blood vessel.

26. The micro-generator implant device of claim 25, wherein said first mechanism includes a rotating mechanism for rotating within said blood vessel.

27. The micro-generator implant device of claim 25, wherein said first mechanism includes a leaf for absorbing energy from said flowing of blood through said blood vessel.

28. The micro-generator implant device of claim 25, wherein said first mechanism includes a flexible membrane, operatively connected to a wall of said blood vessel, for absorbing energy from said flowing of blood through said blood vessel.

29. The micro-generator implant device of claim 28, wherein said flexible membrane is disposed within said blood vessel.

30. The micro-generator implant device of claim 28, wherein said second mechanism is disposed outside of said blood vessel.

31. The micro-generator implant device of claim 1, wherein said micro-generator is disposed within a limb of said living body.

32. The micro-generator implant device of claim 1, wherein said limb includes a prosthesis device.

33. The micro-generator implant device of claim 15, wherein said micro-generator is operatively connected with said stent, said stent disposed within a blood vessel.

34. The micro-generator implant device of claim 33, wherein said stent is a cylindrical stent.

35. The micro-generator implant device of claim 33, wherein said stent is a coil stent.

36. A method of generating energy within a living body, the method comprising the steps of:
(a) providing a micro-generator implant device including:
(i) a micro-generator, disposed within a living body, said micro-generator including:
(A) a first mechanism for harnessing mechanical energy from a natural body movement, and
(B) a second mechanism for converting said mechanical energy to electrical energy, and
(b) generating energy within the living body by means of said micro-generator.

37. The method of claim 36, further comprising the step of:
(c) supplying energy to said micro-generator from a natural motion of the body.

38. The method of claim 37, wherein said natural motion of the body includes a motion of heart muscle tissue.

39. The method of claim 37, wherein said natural motion of the body includes a motion of blood passing through a blood vessel.

40. The method of claim 37, wherein said natural motion of the body includes a motion of a limb.
